(19) United States
(12) Patent Application Publication
(10) Pub. No.: US 2009/0005395 A1
(43) Pub. Date: Jan. 1, 2009

(54) SILDENAFIL N-OXIDE AS PRODRUG

(75) Inventors: Lechoslaw A. Turski, Weesp (NL); Axel Stoit, Weesp (NL); Cornelis G. Kruse, Weesp (NL); Sander Vader, Weesp (NL); Martinus Th.M. Tulp, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Solvay Pharmaceuticals, B.V.

(21) Appl. No.: 12/143,915

(22) Filed: Jun. 23, 2008

Related U.S. Application Data
(60) Provisional application No. 60/946,198, filed on Jun. 26, 2007.

(51) Int. Cl. A61K 31/519; C07D 487/04; A61P 15/10; A61P 9/12

(52) U.S. Cl. 514/252.16; 544/262

(57) ABSTRACT

Embodiments of the present invention relate to a compound of formula (1^{A})

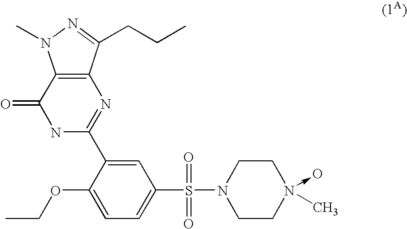

or a pharmacologically acceptable salt, hydrate, or solvate thereof. Other embodiments of the present invention relate to a pharmaceutical composition containing this compound, to methods for preparing this compound, and to methods for preparing compositions containing this compound. Yet other embodiments of the invention relate to uses of this compound, and compositions containing it, for the manufacture of medicaments and pharmaceutical compositions for treating erectile dysfunction or pulmonary arterial hypertension.

This application claims the benefit of priority of U.S. Provisional Application No. 60/946,198, filed on Jun. 26, 2007, the disclosure of which is incorporated herein by reference.

SILDENAFIL N-OXIDE AS PRODRUG

| INDEX | page |
|---|---|
| Title of the invention | 1 |
| Index | 1 |
| Description | 6 |
| Definitions | 11 |
| Example 1: Analytical methods | 15 |
| Example 2: Syntheses of specific compounds | 18 |
| Example 3: Pharmacological methods | 19 |
| Example 4: Pharmacokinetic and pharmacological test results | 20 |
| Example 5: Pharmaceutical preparations | 25 |
| Bibliography | 29 |
| Claims | 30 |
| Abstract | 34 |

This invention relates to the fields of pharmaceutical and organic chemistry. Embodiments of the present invention relate to a sildenafil N-oxide compound, 1-[[3-(6,7-dihydro-1-methyl-7-oxo-3-propyl-1H-pyrazolo[4,3-d]pyrimidin-5-yl)-4-ethoxyphenyl]sulfonyl]-4-methyl-4-oxido-piperazine, of formula (1^{A}):

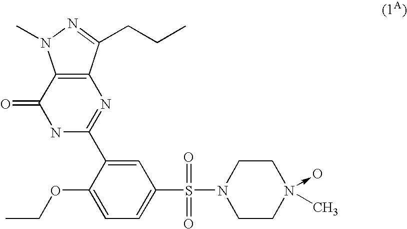

as well as pharmaceutical compositions comprising this compound, methods for preparing this compound, and methods for preparing compositions comprising this compound.

Sildenafil, a compound of formula (1) provided below, is a potent and selective inhibitor of cGMP specific phosphodiesterase type 5 (PDE-V)

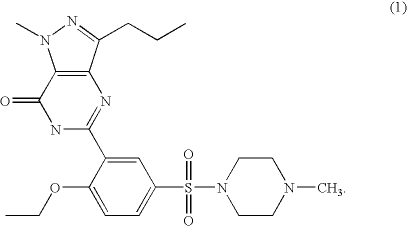

### 1-[4-ethoxy-3-(6,7-dihydro-1-methyl-7-oxo-3-propyl-1H-pyrazolo[4,3-d]pyrimidin-5-yl)phenyl-sulfonyl]-4-methylpiperazine, sildenafil (EP 0 463 756)

Studies in vitro have shown that sildenafil is selective for PDE-V. Its effect is more potent on PDE-V than on other known phosphodiesterases (10-fold for PDE-VI, >80-fold for PDE-1, >700-fold for PDE-II, PDE-III, PDE-IV, PDE-VII, PDE-VIII, PDE-IX, PDE-X, and PDE-XI). The approximately 4.000-fold selectivity for PDE-V versus PDE-III is important because PDE-III is involved in control of cardiac contractility. Sildenafil is only about 10-fold as potent for PDE-V compared to PDE-VI, an enzyme found in the retina which is involved in the phototransduction pathway of the retina. This lower selectivity is thought to be the basis for abnormalities related to color vision observed with higher doses or plasma levels.

Sildenafil is cleared predominantly by the CYP3A4 (major route) and CYP2C9 (minor route) hepatic microsomal iso-enzymes. The major circulating metabolite of sildenafil results from N-demethylation of sildenafil. This metabolite, also known as UK 103,320, has a PDE selectivity profile similar to sildenafil and an in vitro potency for PDE-V approximately 50% of the parent drug. Plasma concentrations of this metabolite are approximately 40% of those seen for sildenafil so the metabolite accounts for about 20% of sildenafil's pharmacologic effects.

N-oxides have been known since 1894. By now it is very well known that N-oxides are metabolites of many tertiary amines, and in most cases are also intermediates between tertiary amines and their N-dealkylated analogs. Most, but not all, tertiary amine drugs give rise to N-oxides. This is the case with morphine, imipramine, promazine, cinnarizine and nicotine. The amount of N-oxidation that occurs varies from trace amounts to a nearly quantitative conversion. Some N-oxides were shown to be more potent than their corresponding tertiary amines. The most famous example of this is chlordiazepoxide (Librium®), one of the most frequently used drugs in psychiatric and general medicine. In many more cases however, N-oxides were found to be less potent than their corresponding tertiary amines, and N-oxidation is most commonly regarded to be metabolic deactivation. While N-oxides are easily reduced to their corresponding tertiary amines by chemical means, in the human body this conversion occurs in varying degrees. Some N-oxides undergo nearly quantitative reductive conversion to their corresponding tertiary amines and in other cases the conversion is a mere trace reaction or even completely absent (Bickel, 1969). Thus, the formation of N-oxides and their corresponding tertiary amines is unpredictable. N-oxides may or may not be reduced to their corresponding tertiary amines. When N-oxides are converted to their corresponding tertiary amines, the conversion may be in mere trace amounts or nearly quantitative. Further, once formed, N-oxides may be more active than their corresponding tertiary amines, less active or even completely inactive.

It is generally accepted that therapeutic as well as toxic effects of drugs are related to their concentration at the relevant target sites. Because generally speaking the latter are not easily accessible, blood plasma levels are used as approximations of relevant drug concentrations. During drug development a window of suitable plasma concentrations are defined providing a lower limit or range for efficacy, and an upper range at which side effects start to become apparent. In ideal situations, the two concentrations are so far apart that it is easy to administer the drug in such a way that it is effective, yet does not give rise to side effects. In reality, situations are hardly ever ideal, and most drugs show side effects. In most cases, the occurrence of side effects can be linked to peak plasma concentrations exceeding the lower level associated with the occurrence of side effects.

Sildenafil produces peak plasma concentrations resulting in side effects. The most commonly observed adverse events associated with the use of sildenafil include sneezing, headache, flushing, palpitations, dyspepsia, increased intraocular pressure, blurred vision, photophobia, severe hypotension, ventricular arrhythmias, myocardial infarction, stroke, and priapism. These adverse effects can significantly limit the dose level, frequency, and duration of drug therapy. Adverse events can be attenuated using special formulations, but different compounds can solve the problem, too.

The objective of the present invention is to find a compound with the advantages of sildenafil while avoiding its disadvantages. Generally, prodrugs have an identical pharmacological profile, but a more favorable pharmacokinetic profile.

### DESCRIPTION

It was found that sildenafil-N-oxide, a compound of formula (1^{A}), acts as a prodrug, and when it is administered orally it is rapidly converted to sildenafil, a compound of formula (1), and to N-desmethylsidenafil, a compound of formula (2), an active metabolite of sildenafil, as shown below.

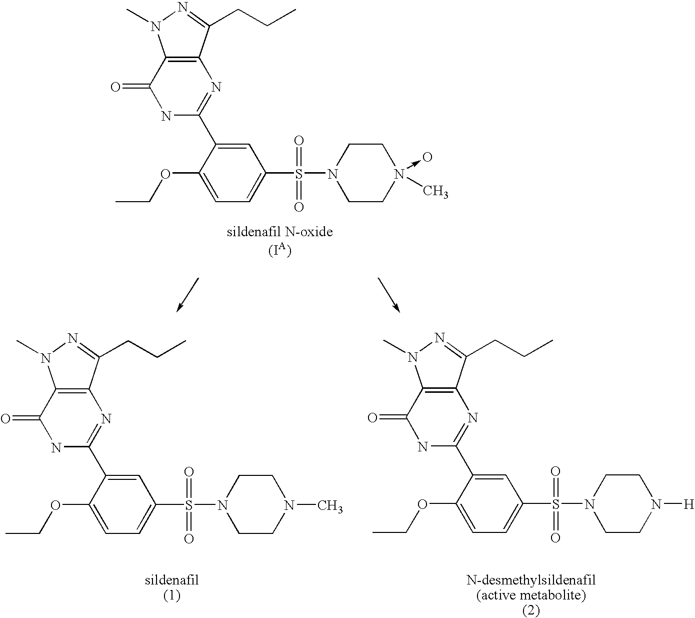

Embodiments of the invention relate to a compound of formula (1^{A}), sildenafil N-oxide, or a pharmacologically acceptable salt, hydrate or solvates thereof. In some embodiments, the invention relates to a compound of formula (1^{A}), which may be substantially free of sildenafil, a compound of formula (1), or a pharmacologically acceptable salt, hydrate or solvate thereof. Sildenafil N-oxide can be prepared by oxidizing sildenafil with a suitable oxidizing agent, for instance m-CPBA. Pharmaceutically acceptable salts may be obtained using standard procedures well known in the art, for example by mixing a compound of the present invention with a suitable acid, for instance an inorganic acid or an organic acid.

Sildenafil N-oxides and compositions comprising them are useful in treating conditions or diseases effectively treatable—albeit with side effects—with sildenafil such as erectile dysfunction (impotence) and pulmonary arterial hypertension (PAH).

Other embodiments of the invention include, but are not limited to:

pharmaceutical compositions for treating, for example, a disorder or condition treatable by sildenafil such as erectile dysfunction or pulmonary arterial hypertension, the compositions comprising sildenafil N-oxide (a compound of formula 1^{A}) or a pharmaceutically acceptable salt, hydrate, or solvate thereof, and at least one pharmaceutically acceptable carrier and/or at least one pharmaceutically acceptable auxiliary substance; and

methods of treating a disorder or condition treatable by sildenafil such as erectile dysfunction or pulmonary arterial hypertension, and methods comprising administering a composition comprising a therapeutically effective amount of sildenafil N-oxide (a compound of formula 1^{A}), or a pharmaceutically acceptable salt, hydrate, or solvate thereof, to a human or animal patient in need of such treating.

Yet other embodiments of the invention relate to the use of sildenafil N-oxide (a compound of formula 1^{A}), or a pharmaceutically salt, hydrate, or solvate thereof, for the manufacture of medicament or a pharmaceutical composition.

The invention further relates to combination therapies wherein a compound comprising sildenafil N-oxide (a compound of formula 1^{A}), or a pharmaceutically acceptable salt, hydrate, or solvate thereof, or a pharmaceutical composition or formulation comprising sildenafil N-oxide (a compound of formula 1^{A}), is administered concurrently or sequentially or as a combined preparation with another therapeutic agent or agents, for instance sildenafil or N-demethylsildenafil, for treating one or more of the conditions listed herein. Such other therapeutic agent(s) may be administered prior to, simultaneously with, or following the administration of the compounds of the invention.

Embodiments of the invention also relate to compounds, pharmaceutical compositions, kits and methods for treating a disorder or condition treatable by sildenafil, the method comprising administering to a patient in need of such treating sildenafil N-oxide (a compound of formula (1^{A}), or a pharmaceutically acceptable salt, hydrate, or solvate thereof.

The invention also provides methods of preparing the compounds of the invention and the intermediates used in those methods.

Some of the crystalline forms for the compounds may exist as polymorphs, and as such, are intended to be included in the present invention. In addition, some of the compounds may form solvates with water (i.e., hydrates), or common organic solvents. Such solvates also fall within the scope of this invention.

Isotopically-labeled sildenafil N-oxide, or pharmaceutically acceptable salts thereof, detectable by PET or SPECT, also fall within the scope of the invention. The same applies to compounds of formula (1^{A}) labeled with [¹³C]—, [¹⁴C]—, [³H]—, [¹⁸F]—, [¹²⁵I]— or other isotopically enriched atoms, suitable for receptor binding or metabolism studies.

Discovering that sildenafil N-oxide is useful as a prodrug offers possibilities of the use of these compounds as alternative to the use of sildenafil, with the clinical benefits of an extended duration of action and a blunted peak plasma concentration, leading to an enhanced side-effect profile. Thus in some embodiments of the present invention, compounds of the present invention may be substantially free of sildenafil, 1-[4-ethoxy-3-(6,7-dihydro-1-methyl-7-oxo-3-propyl-1H-pyrazolo[4,3-d]pyrimidin-5-yl)phenylsulfonyl]-4-methylpiperazine. Within the context of the present invention, “substantially free” means that compound of the present invention contains less than about 50%, 40%, 30%, 20%, 10%, 1%, 0.5% or is, within detectable limits, free of sildenafil as an impurity. Pharmaceutical compositions comprising sildenafil N-oxide which are substantially free of sildenafil are within the scope and spirit of the present invention.

### DEFINITIONS

Any compound metabolized in vivo to provide the bioactive agent (i.e., sildenafil) is a prodrug within the scope and spirit of the application. Prodrugs are therapeutic agents, inactive per se, but transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (Bundgaard, 1985; King, 1994; Stella, 2004; Ettmayer, 2004; Järvinen, 2005).

The term “polymorphism” is defined as the ability of a compound to exist in more than one crystal form, a so-called polymorph. Polymorphism is a frequently occurring phenomenon. Polymorphism is affected by several crystallization conditions such as temperature, level of supersaturation, the presence of impurities, polarity of solvent, rate of cooling. Polymorphs can be characterized by several methods such as solid state NMR, solubility tests, DSC or melting point determination, IR or Raman spectroscopy.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. Throughout the description and the claims of this specification the word “comprise” and variations of the word, such as “comprising” and “comprises” is not intended to exclude other additives, components, integers or steps. The term “composition” as used herein encompasses a product comprising specified ingredients in predetermined amounts or proportions, as well as any product that results, directly or indirectly, from combining specified ingredients in specified amounts. In relation to pharmaceutical compositions, this term encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. The pharmaceutical composition includes enough of the active object compound to produce the desired effect upon the progress or condition of diseases. Accordingly, the pharmaceutical compositions of the present invention encompass any composition made by admixing a compound of the present invention and a pharmaceutically acceptable carrier.

Within the context of this application, the term “combination preparation” comprises both true combinations, meaning sildenafil N-oxide or a pharmaceutically acceptable salt, hydrate, or solvate thereof, and other medicaments physically combined in one preparation such as a tablet or injection fluid, as well as “kit-of-parts”, comprising sildenafil N-oxide or a pharmaceutically acceptable salt, hydrate, or solvate thereof, and sildenafil or another medicament in separate dosage forms, together with instructions for use, optionally with further means for facilitating compliance with the administration of the component compounds, e.g., label or drawings. With true combinations, the pharmacotherapy by definition is simultaneous. The contents of “kit-of-parts,” can be administered either simultaneously or at different time intervals. Therapy being either concomitant or sequential will be dependant on the characteristics of the other medicaments used, characteristics like onset and duration of action, plasma levels, clearance, etc., as well as on the disease, its stage, and characteristics of the individual patient.

By “pharmaceutically acceptable,” it is meant the carrier, diluent or excipient must be compatible with the other ingredients of the formulation and not deleterious to the recipient thereof.

Dose, as used herein, is the same as the recommended treatment dose for sildenafil, 50 mg, as well as lower and higher dosages as appropriate. Pharmacokinetic, pharmacodynamic, and other considerations may alter the dose actually administered to a higher or lower value. The dose of the compound to be administered will depend on the relevant indication, the age, weight, and sex of the patient and may be determined by a physician. The dosage will be in the range of from 0.01 mg/kg to 10 mg/kg. The typical daily dose of the active ingredients varies within a wide range and will depend on various factors such as the relevant indication, the route of administration, the age, weight, and sex of the patient and may be determined by a physician. In general, oral and parenteral dosages will be in the range of 0.1 to 1,000 mg per day of total active ingredients.

The term “therapeutically effective amount” as used herein refers to an amount of a therapeutic agent to treat or prevent a condition treatable by administrating a composition of the invention. That amount is the amount sufficient to exhibit a detectable therapeutic, preventative, or ameliorative response in a tissue system, animal, or human. The effect may include, for example, treating or preventing the conditions listed herein. The precise effective amount for a subject will depend upon the subject's size and health, the nature and extent of the condition being treated, recommendations of the treating physician (researcher, veterinarian, medical doctor or other clinician), and the therapeutics, or combination of therapeutics, selected for administration. Thus, it is not useful to specify an exact effective amount in advance. The term “pharmaceutically acceptable salt” refers to those salts that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They can be prepared in situ when finally isolating and purifying the compounds of the invention, or separately by reacting them with pharmaceutically acceptable non-toxic bases or acids, including inorganic or organic bases and inorganic or organic acids. The term “treatment” as used herein refers to any treatment of a mammalian, preferably human condition or disease, and includes: (1) preventing the disease or condition from occurring in a subject predisposed to the disease, but not yet diagnosed as having it, (2) inhibiting the disease or condition, i.e., arresting its development, (3) relieving the disease or condition, i.e., causing the condition to regress, or (4) stopping the symptoms of the disease. As used herein, the term “medical therapy” intendeds to include prophylactic, diagnostic, and therapeutic regimens carried out in vivo or ex vivo on humans or other mammals. The term “subject” as used herein, refers to an animal, for example, a mammal, such as a human, who has been the object of treatment, observation or experiment.

### EXAMPLE 1

### Analytical Methods

Nuclear magnetic resonance spectra (¹H NMR and ¹³C NMR, APT) were determined in the indicated solvent using a Bruker DRX 600 (¹H: 600 MHz, ¹³C: 150 MHz) at 300 K, unless indicated otherwise. The spectra were determined in deuterated DMSO, obtained from Cambridge Isotope Laboratories Ltd. Chemical shifts (δ) are given in ppm downfield from tetramethylsilane (1H). Coupling constants J are given in Hz. Peak shapes in the NMR spectra are indicated with the symbols ‘q’ (quartet), ‘dq’ (double quartet), ‘t’ (triplet), ‘dt’ (double triplet), ‘d’ (doublet), ‘dd’ (double doublet), ‘s’ (singlet), ‘bs’ (broad singlet) and ‘m’ (multiplet). NH and OH signals were identified after mixing the sample with a drop of D₂O.

Melting points were recorded on a Büchi B-545 melting point apparatus.

Flash chromatography refers to purification using the indicated eluens and silica gel (either Acros: 0.030-0.075 mm or Merck silica gel 60: 0.040-0.063 mm).

Reactions were monitored by using thin-layer chromatography (TLC) on silica coated plastic sheets (Merck precoated silica gel 60 F254) with the indicated eluens. Spots were visualized by UV light (254 nm) or I₂.

Liquid Chromatography-Mass Spectrometry (LC-MS)

The LC-MS system consists of 2 Perkin Elmer series 200 micro pumps. The pumps were connected to each other by a 50 μl tee mixer, connected to a Gilson 215 auto sampler. The method was as follows:

| step | total time | flow (μl/min) | A (%) | B (%) |
|---|---|---|---|---|
| 0 | 0 | 2000 | 95 | 5 |
| 1 | 1.8 | 2000 | 0 | 100 |
| 2 | 2.5 | 2000 | 0 | 100 |
| 3 | 2.7 | 2000 | 95 | 5 |
| 4 | 3.0 | 2000 | 95 | 5 |

A = 100% Water with 0.025% HCOOH and 10 mmol NH₄HCOO pH = +/−3
B = 100% ACN with 0.025% HCOOH

The auto sampler had a 2 μl injection loop. The auto sampler was connected to a Waters Atlantis C18 30*4.6 mm column with 3 μm particles. The column was thermo stated in a Perkin Elmer series 200 column oven at 40° C. The column was connected to a Perkin Elmer series 200 UV meter with a 2.7 μl flowcel. The wavelength was set to 254 nm. The UV meter was connected to a Sciex API 150EX mass spectrometer. The mass spectrometer had the following parameters:

Scan range: 150-900 a.m.u.; polarity: positive; scan mode: profile; resolution Q1: UNIT; step size: 0.10 a.m.u.; time per scan: 0.500 sec; NEB: 10; CUR: 10 IS: 5200; TEM: 325; DF: 30; FP: 225 and EP: 10. The light scattering detector was connected to the Sciex API 150. The light scattering detector was a Sedere Sedex 55 operating at 50° C. and 3 bar N₂. The complete system was controlled by a G3 powermac.

Sildenafil and its N-oxide were analyzed in mouse plasma and brain samples using a generic bioanalytical method comprising protein precipitation and HPLC with MS/MS detection.

Sample preparation: Proteins in 100 μl plasma were precipitated with acetonitrile, and 5 μl samples of the obtained solution were analyzed. Complete brains were homogenized and centrifuged, and 10 μl samples of the supernatant were analyzed.

Liquid Chromatography-Tandem Mass Spectrometry (LC-MS/MS) was performed using a Sciex API4000 LC-MS/MS. Quantification of the samples was done using extracted calibration samples, treated the same as the study samples in the range of 1-5000 ng/ml and 5.0-5000 ng/brain for plasma and brains samples, respectively. Compound peak area was used for quantification. The calibration curves were fitted to the model y=A+Bx+Cx² (y is the peak area of the analyte, x is the nominal calibration level in ng/ml (plasma) or ng/g (brain), A is the intercept, B is the slope and C is the description of the curvature). 1/x² weighing was used. LC-MS/MS system performance was monitored using a reference solution injected at standard intervals. The method use for testing was not validated in detail, therefore the reported concentrations were good estimations. The Lower Limit Of Quantification (LLOQ) was established at 1.00 ng/ml and 5.00 ng/brain, for plasma and brain samples, respectively. Reversed phase HPLC was performed using gradient elution by a Hypersil BDS C18 100×4.6 mm 3 μm analytical column, with a temperature of 45° C. and a flow of 1.00 ml/min:

Solvent

| Time (min) | % A | % B | % C | % D |
|---|---|---|---|---|
| 0.00 | 10.0 | 40.0 | 50.0 | 0.0 |
| 1.00 | 10.0 | 40.0 | 50.0 | 0.0 |
| 2.00 | 10.0 | 10.0 | 80.0 | 0.0 |
| 4.00 | 10.0 | 10.0 | 80.0 | 0.0 |
| 4.10 | 10.0 | 40.0 | 50.0 | 0.0 |
| 7.00 | 10.0 | 40.0 | 50.0 | 0.0 |

| | |
|---|---|
| Solvent A | 100 mM NH₄FA/1% FA |
| Solvent B | Milli-Q water |
| Solvent C | methanol |
| Solvent D | acetonitrile |

Detection on MS/MS was done using positive MRM ionization. The following ions were measured:

| | sildenafil | N-oxide sildenafil |
|---|---|---|
| Q1 | 475.3 | 491.4 |
| Q3 | 100.1 | 99.2 |

### EXAMPLE 2

### Syntheses of Specific Compounds

1-[[3-(6,7-dihydro-1-methyl-7-oxo-3-propyl-1H-pyrazolo[4,3-d]pyrimidin-5-yl)-4-ethoxy-phenyl]sulfonyl]-4-methylpiperazine (sildenafil), and 1-[[3-(6,7-dihydro-1-methyl-7-oxo-3-propyl-1H-pyrazolo[4,3-d]pyrimidin-5-yl)-4-ethoxy-phenyl]sulfonyl]-piperazine (N-desmethylsildenafil) were synthesized as in EP 0 463 756.

1-[[3-(6,7-dihydro-1-methyl-7-oxo-3-propyl-1H-pyrazolo[4,3-d]pyrimidin-5-yl)-4-ethoxyphenyl]sulfonyl]-4-methyl-4-oxido-piperazine (sildenafil N-oxide) was prepared as follows:

Free base of sildenafil (1.42 g, 3 mmol) was dissolved in 75 ml dichloromethane and cooled to −10° C. In small portions, meta-chloroperbenzoic acid (m-CPBA, 0.74 g, 3 mmol, 70% in H₂O) was added to the mixture, and the solution was stirred at −10° C. for 2.5 hours. Solid K₂CO₃ (2 g) was added, and the resulting mixture was stirred for another 30 minutes at 0° C. The reaction mixture was filtrated (glass funnel), and the precipitate was washed with DCM. The resulting solution was concentrated, yielding the title compound as a solid (1.36 g, 92%). M.p.: >200° C. (decomposition). LCMS; Rt: 1.21 min, ([M+H]⁺=491). ¹H—NMR (600 MHz, D₆DMSO): δ 7.89-7.85 (m, 2H), 7.39 (d, J=8 Hz, 1H), 4.18 (q, J=7 Hz, 2H), 4.16 (s, 3H), 3.51-3.43 (m, 4H), 3.04 (s, 3H), 3.02-2.96 (m, 2H), 2.93-2.88 (m, 2H), 2.78 (t, J=7 Hz, 2H), 1.77-1.70 (m, 2H), 1.30 (t, J=7 Hz, 3H), 0.93 (t, J=7 Hz, 3H).

### EXAMPLE 3

### Pharmacological Methods

In vitro inhibition of phosphodiesterases was measured at CEREP (128, rue Danton, 92500 Rueil-Malmaison, France), using well documented procedures: PDE-I from bovine brain (Nicholson, 1989), PDE-II from human U-937 cells (Torphy, 1992), PDE-III from human platelets (Weishaar, 1986), pDE-IV from Human U937 monocytes (Torphy, 1992), PDE-V from human platelets (Weishaar, 1986) and PDE-VI from bovine retina (Ballard, 1998).

The human colon model TIM2 (TNO Intestinal Model 2) is a dynamic model for the human large intestine that simulates in vivo conditions. It is an artificial digestive system that has been validated by many studies (Minekus, 1999).

Sildenafil N-oxide, as well as the pharmacologically acceptable salts, hydrates, and solvates thereof, are prodrugs. They are useful in the treatment of diseases effectively treatable—albeit with side effects—with sildenafil such as erectile dysfunction (impotence) and pulmonary arterial hypertension

### EXAMPLE 4

### Pharmacokinetic and Pharmacological Test Results

Sildenafil and its N-oxide were individually administered (either intravenously (i.v.) or orally (p.o.)) to male NMRI mice (3 animals per time point), after which their plasma and brain were analyzed by LC-MS (see method described above) for both compounds. Data was averaged (n=3), and the results obtained are provided in Table 1 below.

TABLE 1
Plasma and brain concentrations of sildenafil and its N-oxide.

| | | Sildenafil | | Sildenafil-N-oxide | |
|---|---|---|---|---|---|
| | | Plasma | Brain | Plasma | Brain |
| Administered | Time (h) | [ng/ml] | [ng/g] | [ng/ml] | [ng/g] |
| Sildenafil 1.0 mg/kg | 0.17 | 343 | 125 | 3.0 | 0 |
| i.v. | 0.5 | 143 | 40 | 2.1 | 0 |
| | 1 | 52 | 20 | 0.67 | 0 |
| | 3 | 2.4 | 1.2 | 0.61 | 0 |
| | 7 | 0.34 | 0 | 0 | 0 |
| | 24 | 0 | 0 | 0 | 0 |
| Sildenafil | 0.17 | 533 | 155 | 8.0 | 0 |
| 10 mg/kg p.o. | 0.5 | 457 | 129 | 5.3 | 0 |
| | 1 | 260 | 93 | 4.0 | 0 |
| | 3 | 32 | 13 | 0.48 | 0 |
| | 7 | 5.6 | 3.1 | 0.15 | 0 |
| | 24 | 0 | 0 | 0 | 0 |
| Sildenafil-N- | 0.17 | 14 | 8.6 | 225 | 125 |
| oxide 1.0 mg/kg | 0.5 | 6.1 | 3.3 | 49 | 28 |
| i.v. | 1 | 8.8 | 1.8 | 17 | 1.8 |
| | 3 | 3.9 | 1.6 | 0.81 | 2.7 |
| | 7 | 0.31 | 0 | 0 | 0 |
| | 24 | 0 | 0 | 0 | 0 |
| Sildenafil-N- | 0.17 | 7.3 | 5.5 | 1.6 | 92 |
| oxide 10 mg/kg | 0.5 | 52 | 20 | 1.9 | 21 |
| p.o. | 1 | 126 | 38 | 2.7 | 5.5 |
| | 3 | 109 | 37 | 1.4 | 2.7 |
| | 7 | 47 | 20 | 1.1 | 0.9 |
| | 24 | 0 | 0 | 0 | 0 |

When administered to mice (iv. or p.o.), to a marginal extend, sildenafil is metabolized to its N-oxide: The concentration of the N-oxide in the plasma never exceeds 1-2% of that of the parent compound, and in the brain no trace can be found at all. When sildenafil-N-oxide itself is administered, it is reduced to the parent compound, sildenafil. One hour after i.v. administration of sildenafil-N-oxide, sildenafil concentrations in plasma and the brain exceed those of the N-oxide. The effects are more pronounced after oral administration; within half an hour after administration of the N-oxide, sildenafil concentrations in both plasma and the brain are a factor 10 to 100 higher than those of the N-oxide.

Suspended in 1% methylcellulose, sildenafil-N-oxide (1 mg) was inserted into the lumen (120 ml) of the TIM2 model (see above, Minekus, 1999). Samples from the lumen and the dialysate (the latter being a model for the vascular bed of the intestines) were taken at various time intervals, and analyzed for sildenafil-N-oxide and sildenafil. The results are provided in Table 2 below.

TABLE 2
Reduction of sildenafil N-oxide in the human colon model TIM2.

| | |
|---|---|
| Sildenafil-N-oxide | Sildenafil |

| | Lumen | Dialysate | Lumen | Dialysate |
|---|---|---|---|---|
| Time (h) | [ng/ml] | [ng/ml] | [ng/ml] | [ng/ml] |
| 0 | <1.0 | <1.0 | <1.0 | <1.0 |
| 2 | 6.6 | 33 | 11,000 | 130 |
| 4 | 4.7 | <1.0 | 7,800 | 320 |
| 6 | 4.9 | <1.0 | 6,400 | 310 |
| 8 | 6.7 | <1.0 | 5,500 | 280 |
| 24 | 2.7 | <1.0 | 3,300 | 160 |

From the results provided in Table 2 above, it is clear that already within 2 hours after dosing, sildenafil N-oxide was nearly quantitatively reduced to sildenafil. Because many studies validated TIM2 as an in vitro model with high predictive value for the gastrointestinal conditions in living human beings, it is predicted that also in man, after oral administration, sildenafil N-oxide will be reduced to sildenafil. Thus, sildenafil N-oxide is a prodrug.

TABLE 3
Plasma pharmacokinetics of sildenafil and its N-oxide.

| | Sildenafil | | Sildenafil-N-oxide | |
|---|---|---|---|---|
| | Route of administration: | | | |
| | i.v. | p.o. | i.v. | p.o. |
| Dose (mg/kg) | 1 | 10 | 1 | 10 |
| Cₘₐₓ (ng/ml) | 538.5 (C₀)* | 533.3 | 495.1 (C₀)* | 2.7 |
| Tₘₐₓ (hr) | 0.0 | 0.2 | 0.0 | 1.0 |
| t_{1/2} (hr) | 1.4 | 1.1 | 0.4 | 5.1 |
| AUC_{0→end} | 231.6 | 661.1 | 124.9 | 10.8 |
| (ng/ml × hr) | | | | |
| → remark | T_{end} = 7 hrs | T_{end} = 7 hrs | T_{end} = 3 hrs | T_{end} = 24 hrs |
| AUC_{0→∞} | 232.3 | 670.2 | 125.4 | 18.8 |
| (ng/ml × hr) | | | | |
| → remark | T_{end} = ∞ | T_{end} = ∞ | T_{end} = ∞ | T_{end} = ∞ |
| Clearance | 71.8 | — | 132.9 | — |
| (ml/min/kg) | | | | |
| V_{D} (ml/kg) | 8900.0 | — | 5000.0 | — |
| Bioavailability | 28.9 | — | 1.5 | — |
| (%) | | | | |
| Brain/plasma | 0.4 | 0.3 | 0.5 | 2.5 |
| ratio | | | | |

*For i.v. administration, Cₘₐₓ values were extrapolated to T₀ (time zero)

From the data provided in Table 3 above, it is evident that the two compounds, sildenafil and sildenafil N-oxide, have different pharmacokinetic properties.

In a small scale pilot experiment, sildenafil and its N-oxide were individually administered (either intravenously (i.v.) or orally (p.o.)) to male NMRI mice (3 animals per time point), after which their plasma and brain were analyzed by LC-MS (see method described above) for the presence of N-desmethylsildenafil. Data was averaged (n=3), and the results obtained are provided in Table 4 below.

TABLE 4
Plasma and brain concentrations of N-desmethylsildenafil.

N-desmethylsildenafil

| | | Plasma | Brain |
|---|---|---|---|
| Administered | Time (h) | [ng/ml] | [ng/g] |
| sildenafil | 0.17 | 6.0 | <1.0 |
| 1.0 mg/kg i.v. | 0.5 | 8.0 | <1.0 |
| sildenafil | 1 | 92 | 15 |
| 10 mg/kg p.o. | 3 | 25 | 4.2 |
| | 7 | 30 | 0 |
| sildenafil-N-oxide | 0.17 | <1.0 | <1.0 |
| 1.0 mg/kg i.v. | 0.5 | <1.0 | <1.0 |
| sildenafil-N-oxide | 1 | 28 | <1.0 |
| 10 mg/kg p.o. | 3 | 25 | 5.1 |
| | 7 | 24 | 8.2 |

As expected, when administered to mice (iv. or p.o.) sildenafil was metabolized to its N-desmethyl analog, a metabolite that also penetrated the blood-brain barrier, because it was found in brain tissue. Unexpectedly, it was found that when sildenafil-N-oxide was administered, it was also found to be metabolized to N-desmethylsildenafil (only observed after oral dosing).

In order to investigate the activity of sildenafil-N-oxide as inhibitor of different phosphodiesterases, the compound was tested next to sildenafil itself. The results obtained are provided in Table 5 below.

TABLE 5
in vitro inhibition of phosphodiesterases.

| | | | Sildenafil- |
|---|---|---|---|
| | | Sildenafil | N-oxide |
| Enzyme | Source | pIC₅₀ | pIC₅₀ |
| PDE-I | bovine brain | 5.0 | 5.8 |
| PDE-II | human U-937 cells | <5.0 | <5.0 |
| PDE-III | human platelets | 4.5 | <5.0 |
| PDE-IV | human U-937 monocytes | 4.8 | <5.0 |
| PDE-V | human platelets | 7.7 | 6.7 |
| PDE-VI | bovine retina | 7.2 | 6.0 |

For sildenafil, the data in Table 5 confirms what is known from the scientific literature: the compound is a potent inhibitor of PDE-V, and highly selective for this particular subtype, with the exception of PDE-VI, which is also potently inhibited. Sildenafil-N-oxide was found to be 10-fold less potent than sildenafil itself, and showed a comparable selectivity.

In summary, when administrated orally, sidenafil-N-oxide acts as prodrug. It is rapidly converted to the parent compound, sildenafil and to N-desmethylsidenafil, a metabolite of sildenafil reported to have about half its potency. It was also found that sildenafil-N-oxide is not devoid of activity itself. Sildenafil N-oxide has about one tenth of the activity of the parent compound, sildenafil.

### EXAMPLE 5

### Pharmaceutical Preparations

For clinical use, sildenafil N-oxide is formulated into pharmaceutical compositions, which are novel embodiments of the invention because they contain the compounds, for example specific compounds disclosed herein. Types of pharmaceutical compositions that may be used include: tablets, chewable tablets, capsules (including microcapsules), solutions, parenteral solutions, ointments (creams and gels), suppositories, suspensions, and other types disclosed herein, or are apparent to a person skilled in the art from the specification and general knowledge in the art. The active ingredient for instance, may also be in the form of an inclusion complex in cyclodextrins, their ethers or their esters. The compositions are used for oral, intravenous, subcutaneous, tracheal, bronchial, intranasal, pulmonary, transdermal, buccal, rectal, parenteral or other ways to administer. The pharmaceutical formulation contains at least sildenafil N-oxide in admixture with at least one pharmaceutically acceptable adjuvant, diluent and/or carrier. In embodiments of the present invention, the total amount of active ingredients range of from about 0.10% (w/w) to about 95% (w/w) of the formulation, such as from 0.5% to 50% (w/w), and further for example, from 1% to 25% (w/w). In some embodiments, the amount of active ingredient is greater than about 95% (w/w) or less than about 0.1% (w/w).

The compounds of the invention can be brought into forms suitable for administration by means of usual processes using auxiliary substances such as liquid or solid, powdered ingredients, such as the pharmaceutically customary liquid or solid fillers and extenders, solvents, emulsifiers, lubricants, flavorings, colorings and/or buffer substances. Frequently used auxiliary substances include magnesium carbonate, titanium dioxide, lactose, saccharose, sorbitol, mannitol and other sugars or sugar alcohols, talc, lactoprotein, gelatin, starch, amylopectin, cellulose and its derivatives, animal and vegetable oils such as fish liver oil, sunflower, groundnut or sesame oil, polyethylene glycol and solvents such as, for example, sterile water and mono- or polyhydric alcohols such as glycerol, as well as with disintegrating agents and lubricating agents such as magnesium stearate, calcium stearate, sodium stearyl fumarate and polyethylene glycol waxes. The mixture may then be processed into granules or pressed into tablets. A tablet is prepared using the ingredients below:

| Ingredient | Quantity (mg/tablet) |
|---|---|
| Sildenafil N-oxide | 10 |
| Cellulose, microcrystalline | 200 |
| Silicon dioxide, fumed | 10 |
| Stearic acid | 10 |
| Total | 230 |

The components are blended and compressed to form tablets each weighing 230 mg.

The active ingredients may be separately premixed with the other non-active ingredients, before being mixed to form a formulation. The active ingredients may also be mixed with each other, before being mixed with the non-active ingredients to form a formulation.

Soft gelatin capsules may be prepared with capsules containing a mixture of the active ingredients of the invention, vegetable oil, fat, or other suitable vehicle for soft gelatin capsules. Hard gelatin capsules may contain granules of the active ingredients. Hard gelatin capsules may also contain the active ingredients together with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories that contain the active substance mixed with a neutral fat base; (ii) in the form of a gelatin rectal capsule that contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations may be prepared in the form of syrups, elixirs, concentrated drops or suspensions, e.g., solutions or suspensions containing the active ingredients and the remainder consisting, for example, of sugar or sugar alcohols and a mixture of ethanol, water, glycerol, propylene glycol and polyethylene glycol. If desired, such liquid preparations may contain coloring agents, flavoring agents, preservatives, saccharine and carboxymethyl cellulose or other thickening agents. Liquid preparations may also be prepared in the form of a dry powder, reconstituted with a suitable solvent prior to use. Solutions for parenteral administration may be prepared as a solution of a formulation of the invention in a pharmaceutically acceptable solvent. These solutions may also contain stabilizing ingredients, preservatives and/or buffering ingredients. Solutions for parenteral administration may also be prepared as a dry preparation, reconstituted with a suitable solvent before use.

Also provided according to the present invention are formulations and “kits of parts” comprising one or more containers filled with one or more of the ingredients of a pharmaceutical composition of the invention, for use in medical therapy. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration. The use of formulations of the present invention in the manufacture of medicaments for use in treating a condition in which inhibition of phosphodiesterases is required or desired, and methods of medical treatment, comprise the administration of a therapeutically effective total amount of sildenafil N-oxide to a patient suffering from, or susceptible to, a condition in which inhibition of phosphodiesterases is required or desired.

By way of example and not of limitation, several pharmaceutical compositions are given, comprising preferred active compounds for systemic use or topical application. Other compounds of the invention or combinations thereof, may be used in place of (or in addition to) said compounds. The concentration of the active ingredient may be varied over a wide range as discussed herein. The amounts and types of ingredients that may be included are well known in the art.

### BIBLIOGRAPHY

To the extend in which the following references are useful to one skilled in the art, or to more fully describe this invention, they are incorporated herein by reference. Neither these, nor any other documents or quotes cited herein, nor citations to any references, are admitted to be prior art documents or citations.
Ballard et al (1998) J. Urol. 159: 2164
Bickel, M. H.: “The pharmacology and Biochemistry of N-oxides”, Pharmacol. Reviews, 21(4), 325-355, 1969.
Bundgaard, H. (editor), “Design of Prodrugs”, Elsevier, 1985.
EP 0 463 756
Ettmayer, P. et al., “Lessons learned from marketed and investigational prodrugs”, J. Med. Chem., 47, 2393-2404, 2004.
Järvinen, T. et al., “Design and Pharmaceutical applications of prodrugs”, pages 733-796 in: S. C. Gad (editor): “Drug Discovery Handbook”, John Wiley & Sons Inc., New Jersey, U.S.A., 2005.
King, F. D., (editor), page 215 in: “Medicinal Chemistry: Principles and Practice”, 1994, ISBN 0-85186-494-5.
Minekus, M., M. Smeets-Peter, A. Bernalier, S. Marol-Bonnin, R. Havenaar, P. Marteau, M. Alric, G. Fonty, and J. H. J. Huis in't Veld. ‘A computer-controlled system to simulate conditions of the large intestine with peristaltic mixing, water absorption and absorption of fermentation products’. Appl. Microbiol. Biotechnol. 53:108-114, 1999.
Nicholson et al. (1989) Brit. J. Pharmacol. 97: 889-897
Stelia, J., “Prodrugs as therapeutics”, Expert Opin. Ther. Patents, 14(3), 277-280, 2004.
Torphy et al. (1992) J Pharamacol Exp Ther 263: 1195-1205
Weishaar et al. (1986) Biochem. Pharmacol. 35: 787-800

What is claimed is:

1. A compound of formula (1^{A}): 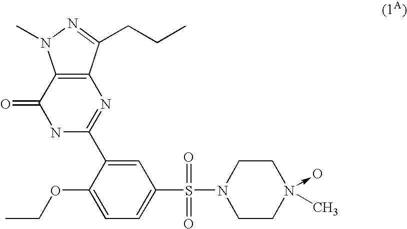
or a pharmacologically acceptable salt, hydrate or solvate thereof.

2. The compound as claimed in claim 1, wherein the compound is substantially free of a 1-[4-ethoxy-3-(6,7-dihydro-1-methyl-7-oxo-3-propyl-1H-pyrazolo[4,3-d]pyrimidin-5-yl)phenylsulfonyl]-4-methyl-piperazine compound, or a pharmacologically acceptable salt, hydrate or solvate thereof.

3. A medicament comprising a compound of formula (1^{A}) 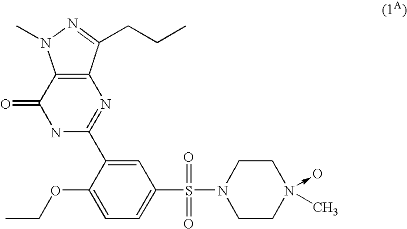
or a pharmacologically acceptable salt, hydrate or solvate thereof.

4. A pharmaceutical composition comprising, at least one pharmaceutically acceptable carrier, at least one pharmaceutically acceptable auxiliary substance, or a combination thereof, and a pharmacologically active amount of at least one compound of formula (1^{A}): 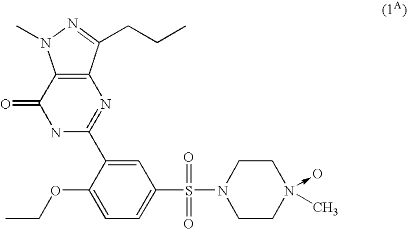
or a pharmacologically acceptable salt, hydrate or solvate thereof.

5. The pharmaceutical composition as claimed in claim 4, wherein the composition is substantially free of a 1-[4-ethoxy-3-(6,7-dihydro-1-methyl-7-oxo-3-propyl-1H-pyrazolo[4,3-d]pyrimidin-5-yl)phenylsulfonyl]-4-methyl-piperazine compound, or a pharmacologically acceptable salt, hydrate or solvate thereof.

6. A combination pharmaceutical preparation comprising (i) a compound of formula (1^{A}): 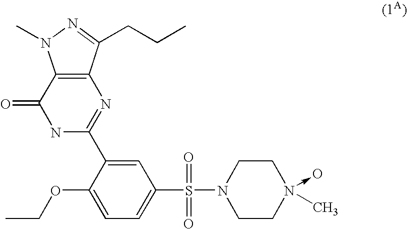
or a pharmacologically acceptable salt, hydrate or solvates thereof, and (ii) a second therapeutic agent, for simultaneous, separate or sequential use in treating erectile dysfunction or pulmonary arterial hypertension.

7. The combination pharmaceutical composition as claimed in claim 6, wherein said second therapeutic agent is sildenafil.

8. A process for preparing a pharmaceutical composition comprising:
(i) combining a compound of formula (1^{A}): 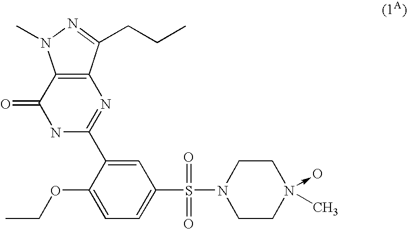
or a pharmaceutically acceptable salt, hydrate, or solvate thereof, with at least one pharmaceutically acceptable carrier or at least one pharmaceutically auxiliary substance, or a combination thereof; and
(ii) formulating the combination produced in (i) into a suitable dosage form.

9. A method of treating erectile dysfunction or pulmonary arterial hypertension, said method comprising administering a composition comprising a therapeutically effective amount of a compound of formula (1^{A}): 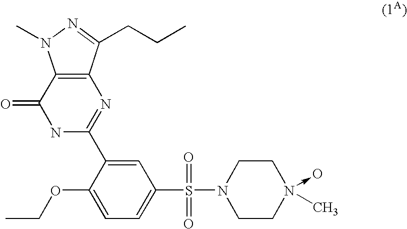
or a pharmaceutically acceptable salt, hydrate, or solvate thereof, to a human or animal patient in need of such treating.

10. A process for preparing a pharmaceutical composition comprising:
(i) combining a compound of formula (1^{A}), 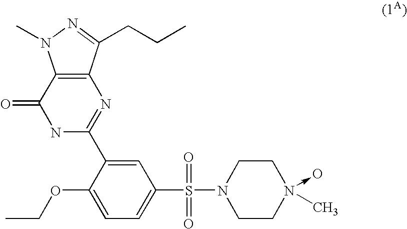
or a pharmaceutically acceptable salt, hydrate, or solvate thereof, a second therapeutic agent, and at least one pharmaceutically acceptable carrier or at least one pharmaceutically auxiliary substance, or a combination thereof; and
(ii) formulating the combination produced in (i) into a suitable dosage form.

11. A process for preparing a compound of formula (1^{A}), the process comprising reacting a compound of formula (1) with an oxidizing agent:

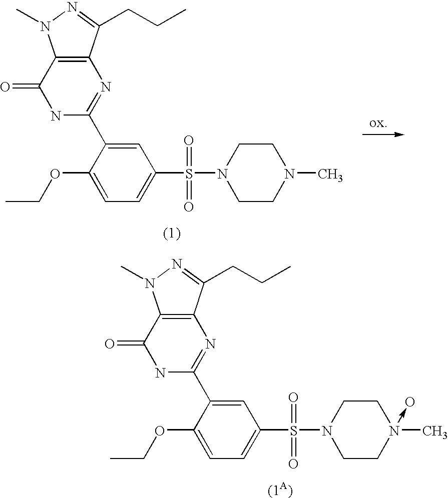

12. The process as claimed in claim 11, wherein said oxidizing agent is m-chloroperbenzoic acid.
